(19) United States
(12) Patent Application Publication
(10) Pub. No.: US 2012/0209245 A1
(43) Pub. Date: Aug. 16, 2012

(54) Process for using a needle having multiple apertures for injections into subcutaneous tissue

(76) Inventors: Greg Yukio Uramoto, Honolulu, HI (US); Brent Yuzo Uramoto, Honolulu, HI (US); Blake Yuzo Uramoto, Honolulu, HI (US)

(21) Appl. No.: 12/932,099

(22) Filed: Feb. 16, 2011

(51) Int. Cl. A61M 5/158

(52) U.S. Cl. 604/506; 604/272

(57) ABSTRACT

The present invention discloses a process for using needle having multiple apertures for insulin injections into subcutaneous tissue, whereby the insulin dispersion pattern is such that insulin is absorbed at an optimal rate even when the needle is inserted too deeply into the muscle layer or when the insulin is injected into lipohypertrophic tissue.

### TECHNICAL FIELD

The present invention relates generally to the use of needles having multiple apertures to perform injections, preferably for medical purposes. The invention relates more specifically to a process for using a needle having at least two holes for injecting insulin into the subcutaneous tissue (hypodermis) of a patient.

### BACKGROUND ART

The skin is an organ that contains many specialized cells and structures. It functions as a protective barrier that interfaces with the environment. There are three layers of the skin—the epidermis, dermis and subcutaneous tissue (hypodermis). The epidermis is the outer layer of skin. The dermis is the middle layer of skin and it contains collagen, elastic tissue and reticular fibers. The bottom layer of skin is called subcutaneous tissue or hypodermis and contains a layer of fat and connective tissue that houses larger blood vessels and nerves. Underneath the subcutaneous tissue is a layer of muscle.

Subcutaneous injections are highly effective in administering vaccines and other medications, such as insulin, morphine, diacetylmorphine or goserelin. Persons with Type 1 diabetes typically inject insulin subcutaneously. This is usually done by pinching a 2-inch fold of skin between ones thumb and index finger before inserting a needle into the pinched up skin.

By way of contrast, intramuscular injections are injections of a substance directly into a muscle and are typically used for particular forms of medication that are administered in small amounts.

Using a conventional hypodermic needle for injections into subcutaneous tissue (but not into the muscle layer) presents several problems for persons who are not medically trained, such as young diabetic patients injecting themselves with insulin. Young diabetic patients often drive hypodermic needles too deeply, past the subcutaneous tissue (fat) layer and into the muscle layer. When insulin is injected intramuscularly, it is not only painful, but is also absorbed too quickly.

Another problem is that, even if intramuscular injections are avoided, young diabetic patients often give themselves multiple insulin injections into the same location on the body, because those injections tend to be less painful due to tissue thickening (lipohypertrophy) as a result of the repeated injections. This is a common side effect of subcutaneous insulin therapy, occurring in up to 50% of patients with type 1 diabetes. See Unn-Brit Johansson et al., Impaired Aborption of Insulin Aspart from Lipohypertrophic Injection Sites, Diabetes Care, vol 28, no. 8, August 2005, at 2025 (incorporated herein by reference). Lipohypertrophic tissue does not absorb insulin effectively. See id. At least one study has found a 25% drop in insulin absorption when injected into lipohypertrophic tissue. See id. Therefore, if absorption of insulin into lipohypertrophic tissue could be optimized, patients could inject themselves into these less painful locations regularly, without excessive loss of insulin absorption efficacy.

Accordingly, it is an object of the present invention to optimize insulin absorption into a patient, even if a needle is driven too deeply, past the subcutaneous tissue and into the muscle layer.

It is a further object of the present invention to optimize insulin absorption at sites that are repeatedly used for insulin injections, so that these less painful sites can be used despite impaired insulin absorption efficacy.

### DISCLOSURE OF THE INVENTION

The above and other objects are achieved by a multi-holed needle that delivers insulin into the subcutaneous layer, even if the end of the needle is inserted into the muscle layer, and produces better distribution and absorption of injected insulin into lipohypertrophic tissue in the subcutaneous layer than a single-holed needle.

Preferably, the multi-holed needle of the present invention comprises a substantially cylindrical shaft having at least two holes: a first hole, having a beveled (diagonally cut) tip with an opening facing a first direction that has a transverse and longitudinal component, located at the distal end of said shaft, and a second hole, with an opening facing a second direction that is diametrically opposite from the transverse component of the first direction, located a separation distance from the first hole (measured between the centers of the first and second holes). Surprisingly, insulin or fluid exiting from the beveled tip opening forms a short and substantially flat cylindrical (coin-shaped) dispersion pattern, and fluid exiting from the second hole opening forms a semi-circular (half-coin shaped) dispersion pattern that is substantially perpendicular to the coin-shaped dispersion pattern. Having both the coin-shaped dispersion pattern and the half-coin shaped dispersion pattern increases the surface area of the injected fluid, so that it can be absorbed by the body at an increased rate.

When the needle is driven too deeply into a patient's tissue such that the first hole (with the beveled tip opening) is driven into the muscle layer, the insulin is still delivered properly into the subcutaneous tissue through the second hole because the muscle layer provides more resistance than the subcutaneous tissue, so that most (if not all) of the insulin is deposited into the subcutaneous tissue.

The present invention is operable when the separation distance between the first and second holes (measured between the centers of the first and second holes) is approximately 2 to 5 mm, and optimally approximately 3 mm. The second hole preferably contains an opening that is 0.25 to 0.75 mm in diameter and is substantially rectangular shaped with rounded corners.

The present invention is also a process for using a needle having multiple apertures for injections of fluid, comprising inserting the needle into tissue so that a first hole in a beveled tip (with an opening facing a first direction having a transverse and longitudinal component, located at the distal end of the needle), and a second hole (with an opening facing a second direction that is diametrically opposite the transverse component of the first direction) located a separation distance from the first hole, are both in the subcutaneous layer. Fluid is then injected into the subcutaneous layer through the needle, wherein fluid exiting from the first hole forms a coin-shaped dispersion pattern, and fluid exiting from the second hole forms a half-coin shaped dispersion pattern that is substantially perpendicular to the coin-shaped dispersion pattern. Having both the coin-shaped dispersion pattern and half-coin shaped dispersion pattern increases the surface area of the fluid so that it can be absorbed by a body at an increased rate.

The present invention is also a process for using a needle having multiple apertures for injections of fluid comprising inserting the needle into tissue so that a first hole located at the distal end of the needle is inserted into the muscle layer, and a second hole located a separation distance from the first hole, is inserted into the subcutaneous tissue. Fluid is then injected into the tissue through the needle and exits from the second hole in a half-coin shaped dispersion pattern into the subcutaneous layer. The fluid is then absorbed by the body at an increased rate in the subcutaneous tissue.

For both processes described above, the injections can be made into lipohypertrophic tissue, and the separation distance between the first hole and the second hole of the needle is operably approximately 2 to 5 mm, optimally approximately 3 mm.

The following patents and patent applications may be considered relevant to the field of the invention:

U.S. Pat. No. 5,848,996 to Eldor, incorporated herein by reference, discloses a double-hole pencil point spinal needle composed of a closed end blunt ogival or pencil point tip and two circular coaxial holes in close proximity to the tip. Anesthetic solution may be injected through the coaxial holes in a direction parallel to the long axis of the spinal fluid column which allows an even anesthetic distribution with a low dosage requirement.

U.S. Pat. No. 6,200,296 to Dibisasi, incorporated herein by reference, discloses a needle assembly for medication delivery pen or hypodermic syringe having a usable length of 5 mm that delivers a dose of medication to a proper location in the tissue “strata” or subcutaneous layer, without the need for the user or patient to “pinch-up” the skin layer during the injection.

U.S. Pat. No. 4,411,657 to Galindo, incorporated herein by reference, discloses an improved hypodermic needle to greatly reduce or eliminate trauma to nerve fascicles during the injection of anesthesia. The needle includes a solid, conically shaped, preferably pointed tip, an elongated shaft having a hollow interior for delivering the anesthesia and one or more lateral openings strategically spaced away form the tip of the needle. The pinpoint of the needle separates the nerve fascicles without cutting them.

U.S. Pat. No. 4,898,877 to Massau, incorporated herein by reference, discloses a polymeric hypodermic device that comprises a hollow polymeric needle for injection of substances beneath the skin. The invention provides a novel injection end through which the medication is injected in a direction other than parallel to the axis of the needle, which has had a swirling motion imparted to it, thus lessening trauma tissue and veins.

U.S. Pat. No. 4,737,146 to Amaki et al., incorporated herein by reference, discloses a multi-lumen epidural catheter including a flexible inner tube having a tip opening and a surrounding flexible outer tube having a pair of side openings placed upstream from the tip. An anesthetic solution may be injected through the separate flow and discharge paths defined by this structure to deaden a more elongate zone of a patient in a precisely controlled manner.

### BRIEF DESCRIPTION OF DRAWINGS

FIGS. 1A and 1B are x-ray photographs that depict a conventional single-holed needle (hypodermic needle) injecting fluid past the fat layer into the muscle layer. FIG. 1A shows the needle being driven too deeply into the muscle layer and is taken prior to injection of the fluid and FIG. 1B shows the fluid dispersion pattern into the muscle layer.

FIGS. 1C and 1D are x-ray photographs that depict a double-holed needle according to the present invention, being inserted past the subcutaneous tissue (fat layer) into the muscle layer, but injecting the fluid into the subcutaneous tissue from a second hole located a distance from the end hole. FIG. 1C is taken prior to injection of the fluid and FIG. 1D shows the fluid dispersion pattern into the subcutaneous tissue, with little to no fluid injected into the muscle layer.

FIGS. 2A and 2B are x-ray photographs that depict the process of injecting fluid from a conventional single-holed needle, wherein the fluid that is injected from the end hole into the subcutaneous tissue forms a coin-shape dispersion pattern. FIG. 2A is prior to injection of the fluid and FIG. 2B shows the shape of the fluid dispersion pattern as being a short, uniformly flat cylinder (elliptical coin shape), not a sphere. If the fluid had formed a sphere, the x-ray pattern would be more opaque in the center.

FIGS. 3 to 5 are schematics that depict injections using a double-holed needle into subcutaneous tissue, wherein the needle has a first hole having a beveled tip opening, facing a first direction having a transverse component and a longitudinal component, located at the distal end of the needle. The needle also has a second hole with an opening facing a second direction that is opposite from the transverse component of the first direction, located a separation distance from the first hole.

FIGS. 3A and 3B are top views of the double-holed needle and an injection of that needle into subcutaneous tissue, respectively. FIG. 3B depicts the coin shaped dispersion pattern from the first hole (having a beveled tip opening) and the half-coin shaped dispersion pattern from the second hole's opening, and wherein the half-coin shaped dispersion pattern is substantially perpendicular to the coin shaped dispersion pattern.

FIGS. 4A and 4B are bottom views of the double-holed needle and an injection of that needle into subcutaneous tissue, respectively. FIG. 4A also shows the second hole on the needle. FIG. 4B depicts the bottom view of the same coin shaped and half coined shaped dispersion pattern as shown in FIG. 3B. The half coin shaped dispersion pattern from the second hole is projecting toward the viewer.

FIGS. 5A and 5B are side views of the double-holed needle and an injection of that needle into subcutaneous tissue. FIG. 5A shows the first hole on the opposite side of the second hole. FIG. 5B depicts the side view of the same coin shaped and half coined shaped dispersion pattern as shown in FIG. 3B.

FIGS. 6A and 6B are x-ray photographs that depict the process of injecting fluid from a double-holed needle into subcutaneous tissue. FIG. 6A is taken prior to injection of the fluid and FIG. 6B shows the fluid dispersion pattern once the injection occurs. The fluid dispersion pattern at the end hole forms a coin shaped dispersion pattern (similar to a single holed needle), whereas the fluid dispersion pattern from the second hole forms a half-coin shaped dispersion pattern that is substantially perpendicular to the coin shaped fluid dispersion pattern from the end hole. The uniform opaqueness of the fluid pattern indicates a coin shape from the end hole. The rectangle shaped light pattern indicates a half coin shape from the second hole, viewed from the edge, projecting toward the viewer.

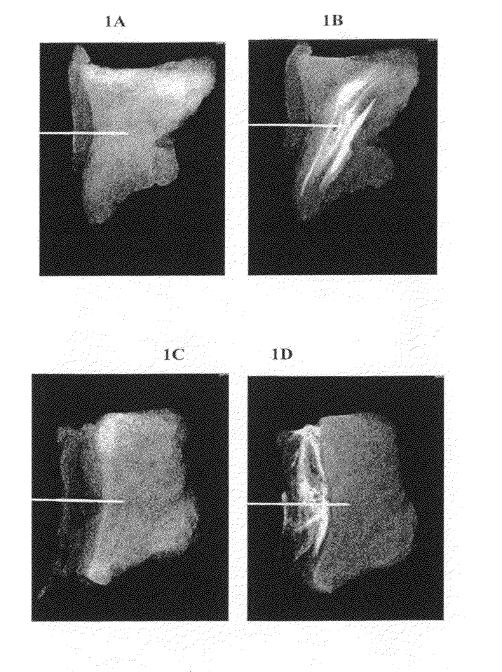

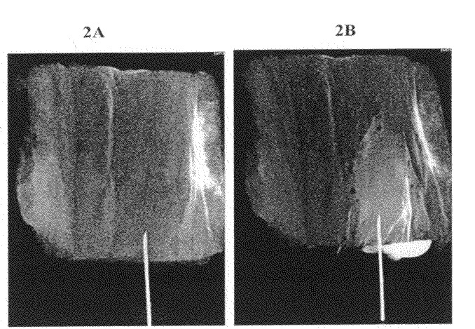

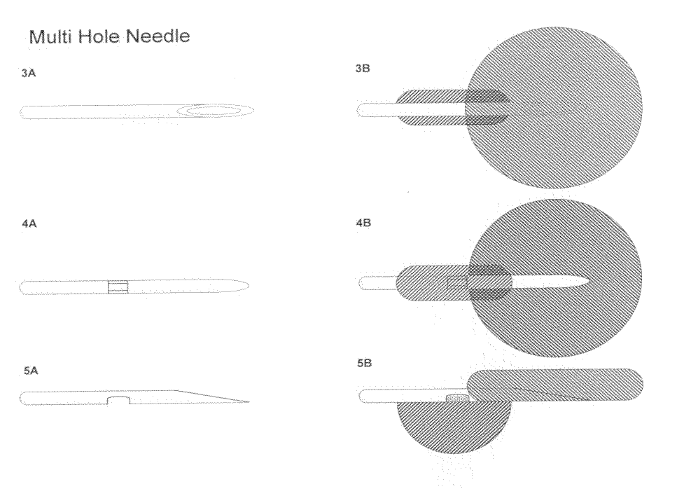

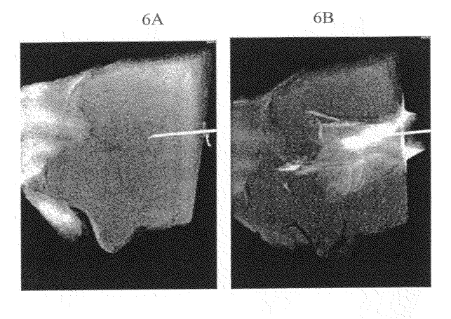

### BEST MODE FOR CARRYING OUT THE INVENTION

The present invention preferably uses a needle having at least two holes. The first hole preferably has a beveled (diagonally cut) tip with an opening facing a first direction having a transverse and longitudinal component and is located at the distal end of the needle. The beveled tip is similar to the tip found on a standard hypodermic needle. To maximize the multi-holed needle's fluid dispersion pattern, the needle preferably has a second hole with an opening facing a second direction that is diametrically opposite from the transverse component of the first direction and this second hole is located a separation distance from the first hole.

Three or more holes can be used, but the multi-holed needle would need to be made out of stronger steel than the conventional needle, because the number of holes adversely affects the structural soundness of the needle. If three or more holes are used, the resultant fluid dispersion pattern would contain multiple half-coin shaped fluid dispersion patterns (see below).

The present invention is operable when the separation distance between the first and second holes (measured from the center of the first and second holes) is approximately 2 to 5 mm, and optimally approximately 3 mm. The second hole preferably contains an opening that is 0.25 to 0.75 mm in diameter and is substantially rectangular shaped with rounded corners.

When the needle is driven too deeply into tissue such that the first hole (with the beveled tip) is driven into the muscle layer, insulin is still delivered properly into the subcutaneous tissue layer through the second hole (and very little of the insulin reaches the muscle layer). This is because the muscle layer provides greater resistance than the subcutaneous tissue layer so that most (if not all) of the insulin is deposited into the subcutaneous tissue layer.

Using a multi-holed needle also maximizes insulin absorption into the subcutaneous tissue, including lipohypertrophic tissue, due to the fluid dispersion pattern that occurs out of the first and second holes. Insulin is dispersed in a short, flat cylindrical (coin) shape out of the first hole (at the distal end of the needle), and a half-coin shape out of the second hole. The plane of the half-coin shape dispersion pattern is substantially perpendicular to the plane of the coin shaped dispersion pattern. This fluid dispersion pattern increases the surface area of the injected fluid, which increases the fluid's absorption rate into tissue, including lipohypertrophic tissue. In fact, a multi-holed needle that disperses a given insulin volume to a 33% larger lipohypertrophic tissue area, may compensate for the reduced insulin absorption of lipohypertrophic tissue. This allows a patient to inject insulin into lipohypertrophic tissue while losing very little insulin efficacy.

While the present invention has been disclosed in connection with the presently preferred best mode described herein, it should be understood that there may be other embodiments which fall within this spirit and scope of the invention as defined by the claims. Accordingly, no limitations are to be implied or inferred in this invention except as specifically and as explicitly set forth in the claims.

### INDUSTRIAL APPLICABILITY

The present invention discloses a process for using a needle having multiple apertures for insulin injections into subcutaneous tissue. However, it can be used for other types of subcutaneous injections that seek to optimize fluid absorption rate through the fluid dispersion pattern.

What is claimed is:

1. A process for using a needle having a beveled tip with an opening facing a first direction having a transverse component and a longitudinal component, defining a first hole located at the distal end of said needle, and a second hole with an opening facing a second direction that is diametrically opposite from said transverse component of said first direction, located a separation distance from said first hole, for injections of fluid comprising:
inserting said needle into tissue so that both of said holes are located in the subcutaneous layer;
injecting fluid into said subcutaneous layer through said needle wherein fluid exiting from said first hole forms a coin-shaped dispersion pattern, and fluid exiting from said second hole forms a half-coin shaped dispersion pattern that is substantially perpendicular to said coin-shaped dispersion pattern;
whereby said coin-shaped dispersion pattern and said half-coin shaped dispersion pattern increase the surface area of said fluid so that it is absorbed by a body at an increased rate.

2. A process for using a needle having a beveled tip with an opening facing a first direction having a transverse component and a longitudinal component, defining a first hole located at the distal end of said needle, and a second hole with an opening facing a second direction that is diametrically opposite from said transverse component of said first direction, located a separation distance from said first hole, for injections of fluid into a subcutaneous layer over a muscle layer comprising:
inserting said needle into tissue so that said first hole is inserted into the muscle layer, and said second hole is inserted into the subcutaneous layer;
injecting fluid into said tissue through said needle wherein said fluid exits from said second hole into said subcutaneous layer in a half-coin shaped dispersion pattern into said subcutaneous layer;
whereby said fluid is absorbed by a body at an increased rate.

3. A process according to claim 1, wherein said tissue is lipohypertrophic tissue.

4. A process according to claim 1, wherein said separation distance is 2 to 5 mm.

5. A process according to claim 1, wherein said separation distance is 3 mm.

6. A process according to claim 2, wherein said tissue is lipohypertrophic tissue.

7. A process according to claim 2, wherein said separation distance is 2 to 5 mm.

8. A process according to claim 2, wherein said separation distance is 3 mm.

9. A needle for injections of fluid into subcutaneous tissue comprising:
a hollow substantially cylindrical shaft having a beveled tip with an opening facing a first direction having a transverse component and a longitudinal component, defining a first hole located at the distal end of said shaft, and having a second hole with an opening facing a second direction that is diametrically opposite from said transverse component of said beveled tip opening, located a separation distance from said first hole;
wherein when said tip is inserted into subcutaneous tissue, fluid exiting from said first hole forms a coin-shaped dispersion pattern, and fluid exiting from said second hole forms a half-coin shaped dispersion pattern that is substantially perpendicular to said coin-shaped dispersion pattern;
whereby said coin-shaped dispersion pattern and said half-coin shaped dispersion pattern increase the surface area of said fluid so that it is absorbed by the body at an increased rate.

10. A needle according to claim 5, wherein said separation distance is 2 to 5 mm.

11. A needle according to claim 5, wherein said separation distance is 3 mm.

12. A needle according to claim 5, wherein said second hole opening is preferably 0.25 to 0.75 mm in diameter, and is substantially rectangular shaped with rounded corners.
